(19) United States
(12) Patent Application Publication
(10) Pub. No.: US 2021/0068730 A1
(43) Pub. Date: Mar. 11, 2021

(54) MEDIUM WITH HYDROPHOBIC PATTERNS AND BREAK LINES DEFINING A BLOOD COLLECTION VOLUME

(71) Applicant: Boston Microfluidics, Inc., Cambridge, MA (US)

(72) Inventors: Brandon T. Johnson, Cambridge, MA (US); Kate E. Christian, Baltimore, MD (US); Russell Grant, Burlington, NC (US); Lachlan Tobiason, Cambridge, MA (US)

(21) Appl. No.: 17/012,191

(22) Filed: Sep. 4, 2020

Related U.S. Application Data
(60) Provisional application No. 62/896,715, filed on Sep. 6, 2019.
(60) Provisional application No. 63/060,279, filed on Aug. 3, 2020.

(51) Int. Cl. A61B 5/15; A61B 5/155

(52) U.S. Cl. CPC A61B 5/150946; A61B 5/150755; A61B 5/150022; A61B 5/155

(57) ABSTRACT

A blood sample collection and/or storage device includes a medium, such as a membrane or microstructured environment for storing a body fluid sample such as a blood sample. The medium has hydrophobic patterns formed thereon or therein to define precisely dimensioned channels for fluid flow or fluid retention. Break lines in the medium defined predetermined areas (or volumes) of the medium. After sample collection, the medium may be broken apart along the break lines to obtain a precisely measured amount of the fluid sample.

### CROSS REFERENCE TO RELATED APPLICATIONS

This application claims priority to a co-pending U.S. Provisional Application Ser. No. 62/896,715 filed Sep. 6, 2019, and to a co-pending U.S. Provisional Application Ser. No. 63/060,279 filed Aug. 3, 2020, each of which is hereby incorporated by reference in its entirety.

### BACKGROUND

### Technical Field

This patent relates to precise collection of body fluids, such as a blood sample.

### Background Information

Blood used for diagnostic testing is most often extracted from a patient with a hypodermic needle and collected in a test tube. The collected blood is then packaged for shipment to a remote lab where various diagnostic tests are performed. However, many diagnostic tests require significantly less volume than the actual collected sample. Separation of cellular components from the sample is also needed for some tests.

Many tests only require small blood samples, where a finger stick rather than a hypodermic needle can produce enough blood. Convenient and widely accessible methods of collecting and preserving small, accurately measured amounts of blood are still needed, however.

### SUMMARY

A medium such as a membrane is used to collect a body fluid sample such as a blood sample. The membrane has hydrophobic patterns to define precisely dimensioned channels for fluid flow. break lines in the membrane defined predetermined areas (or volumes) of the membrane. After collection and transport, the membrane may be broken apart along the break lines to obtain a precisely measured blood sample.

More particularly, in one embodiment a device may include a medium, such as a membrane or microstructured environment, having a channel defined by at least one patterned hydrophobic region. At least one break line intersects the channel to define a predetermined area or collection volume of the medium.

The break lines can be used to define different areas of the medium that can be easily detached for further processing.

In some embodiments, two or more the break lines may define corresponding multiple areas of the medium. The different areas may be coated with different reagents, or may be of differing sizes or shapes.

The hydrophobic region or corresponding regions may define fluid pathways. The pathways can direct fluid samples to different areas, or regulate the fluid's speed of movement, or to encourage further saturation of the medium.

The medium may include multiple layers, some of which may be membranes, and others of which may be lateral flow strips that contain reagents, conjugates, or other materials.

The layers may contain hydrophobic or hydrophilic materials to further direct the fluid.

### BRIEF DESCRIPTION OF THE DRAWINGS

FIGS. 1 to 18 are examples of collection medium which may have channels defined by hydrophobic region and/or precise volumes defined by break lines. In particular:

FIG. 1 shows collection membrane coated with hydrophobic region such as wax;

FIG. 2 shows a similar membrane with break lines;

FIG. 3 is another example where the channel is a rectangle;

FIG. 4 is similar to the FIG. 3 example, but with break lines;

FIG. 5 is a membrane with two parallel channels;

FIG. 6 is an embodiment break lines and without any hydrophobic region patterning;

FIG. 7 is an example where the break lines run lengthwise;

FIG. 8 is an example where the break lines run both lengthwise and across the channel;

FIG. 9 shows an embodiment with break lines formed along the edges of the channel—here the membrane may also be a lateral flow strip held in a hydrophobic housing;

FIG. 10 is an example embodiment where a single channel follows a curved path;

FIG. 11 is an example embodiment similar to FIG. 10 but with break lines.

FIG. 12 is an example embodiment with break lines formed only on certain parts of the sides of the channel;

FIG. 13 is another example embodiment where the break lines follow the curved channel along its length;

FIG. 14 is another embodiment where the membrane has been coated with a substance;

FIG. 15 is an example embodiment having a serpentine channel that runs the length of the membrane, with break lines defining several sections of the serpentine channel;

FIG. 16 is a similar arrangement but without the break lines;

FIG. 17 is another embodiment with break lines in a serpentine channel;

FIG. 18 is a “three-dimensional” implementation where the channel occupies more than one layer;

FIG. 19 is an isometric view of an example blood sample collection device that uses any of the membranes of FIGS. 1-18, before it is used.

FIG. 20 is an exploded view of the device of FIG. 19.

FIG. 21 is an exploded view of a device that has a medium 400 formed of multiple layers of membrane.

FIG. 22 is a medium that includes a channel with multiple branches that feed removable circular areas.

FIG. 23A is an isometric view of another device that uses hydrophobic region to pattern a medium that provides a lateral flow strip.

FIG. 23B is a cross-sectional view of the device of FIG. 23A.

### DETAILED DESCRIPTION OF PREFERRED EMBODIMENTS

This patent application describes a membrane, or other medium such as a microstructured environment, medium that collects and stores precisely defined amounts of a blood or other fluid sample. In general, the medium has one or more channels defined by a wax or other hydrophobic region. In some embodiments, the channels can be defined by creating immiscible hydrophobic regions. Hydrophobic regions in the media can be arranged such that liquids are prevented from entering a region either from hydrophobic forces, via physical occlusion or similar physical barrier.

The one or more channels defined by wax or some other hydrophobic region direct the fluid while it is in the process of being collected along a defined path. These hydrophobic regions can not only be used to define paths but also keep reagents or layers of the medium separate. In addition, the hydrophobic region(s) can be used to define a reaction well where a sample is mixed with a reagent.

The hydrophobic regions may define different types or shapes of fluid pathways. Differently shaped and lengthened paths, such as serpentine or other tortuous paths, may be utilized to regulate and/or slow the speed of fluid movement through or along the medium. Slowing the speed of fluid sample movement may, in turn, allow the medium to more fully absorb the fluid, such as via a resulting slowed capillary action.

The medium may, in some embodiments, be enclosed within several different types of device housings that form a sample collection device.

In some embodiments, sections of the medium may be defined by break lines such as perforations. The sections may outline predetermined area(s) of the medium and/or further define one or more flow path(s). The break lines allow the medium to be subsequently split into sections that have collected predefined volume(s) of the fluid.

The break lines may take the form of different shapes. In one embodiment, break lines in the shape of one or more circles may allow precise volume of a dried body fluid sample, such as a blood sample, to be collected and easily removed from the medium. Currently circular holes are punched out of dried blood spot cards and predefining the circle could aid in automation. break lines can also allow for the easy detachment of a test region from the rest of the device.

In some embodiments break lines may allow for detachment of an assay region as well as a sample region which may then be used for subsequent analysis. In some embodiments break lines may define or control flow rate by narrowing channels. In some embodiments, break lines separate areas of membrane may be treated with different reagents.

The medium may also include a device that provides a microstructured environment. For an environment composed of a number of elements, such as fibers, pores or pillars, arranged in such a way that create a field that slows the flow of specific elements of a fluid such as red cells, white cells or other cellular materials.

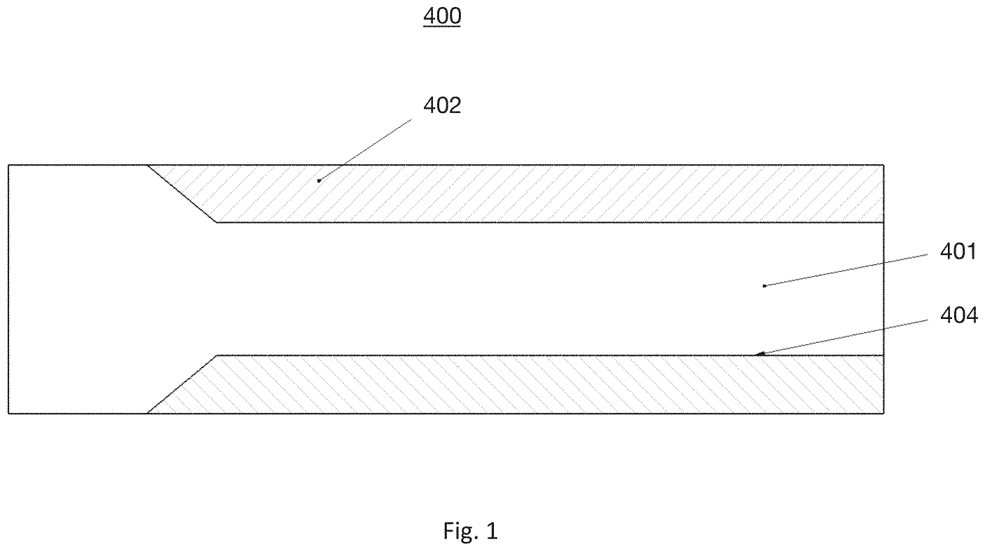

FIG. 1 is a top view of one such medium 400. A primary area 401 contains portions of the membrane through which fluid flows (also referred to herein as the channel 401). That part of the medium 400 through which fluid flows is exposed and uncoated. Other areas 402 are coated with a hydrophobic region, such as wax (and as indicated by hatching in the drawings). The periphery of the hydrophobic region provides a border 404 defining a precise area for the fluid channel 401.

Although only one side of the medium 400 is shown, it should be understood that the hydrophobic region may typically be coated on both face of the medium 400 or fully permeate the membrane 400. In some embodiments a section of medium 400 such as channel 401 may also be partially coated with a hydrophobic region to slow the flow of fluid through this section.

The medium 400 may be planar sheet of a sample medium such as a plasma separation membrane or filter of various types. For example, a mixed-cellulose ester membrane such as the Pall Vivid Plasma Separation available from Pall™ Corporation may be used. The membrane may also be an LF1 glass fiber membrane (sold by General Electric™ Company) or some other medium designed to receive serum or whole blood, which it then separates into a blood portion and a plasma portion.

A membrane-type medium 400 such as LF1 paper has a fibrous structure that causes differential migration of the sample, with a slower rate for red cells, resulting in a gradual separation of plasma sample as it migrates down the channel defined. LF1 paper, which separates plasma from red blood cells through a fiber matrix, is preferred in some embodiments, because it causes a slower migration rate for the blood cells. However other types of separation membranes for blood either liquid or dried may be used for the medium 400. The medium 400 can optionally be previously impregnated with heparin, EDTA, sugars, or other stabilization agents.

Plasma separation may also be achieved through mediums that are non-membrane microstructures that exclude red cells by size. For example, plasma separation can be achieved or enhanced by selectively binding red cells with an agent. Binding agents may typically be coated on a membrane or other micro structures but could also be deposited in a channel. Therefore, it should be understood that other types of microstructures can serve as the medium.

The channel portion of the medium 400 may also be coated with various chemicals to perform a test, such as an assay, on the collected sample.

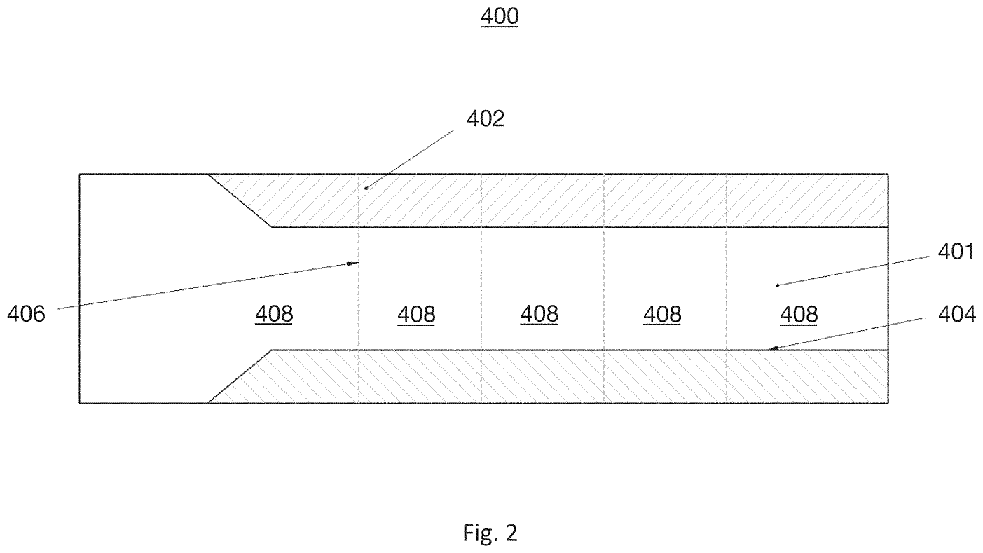

FIG. 2 is an example of a medium 400 having a similar shaped channel. Here, however, break lines 406 (as indicated by the dashed lines) identify individual sections 408 along which the medium 400 may be subsequently broken apart. For example, the initial blood collected may be permitted to be separated, stabilized, and dried on the medium 400. After a time, such as needed to transport the medium 400 to a remote lab, the medium is broken up along the break lines. In this example, the lab would have five (5) different sections 408 of the medium 408 to process. Of course, the medium 400 could have a different number of break lines than shown in FIG. 2, such that the number of sections is less than or greater than five.

The different sections 408 of the medium 400 may serve different purposes. For example, selected sections 408 may be coated with different chemicals to perform different tests, such as an assay, on the cells collected in that section. Thus, a single medium 400 may be used to perform multiple tests and/or apply multiple reagents in the predetermined sections 408.

In other arrangements, the different sections 408 may have different filtering properties, to process different cells of different sizes.

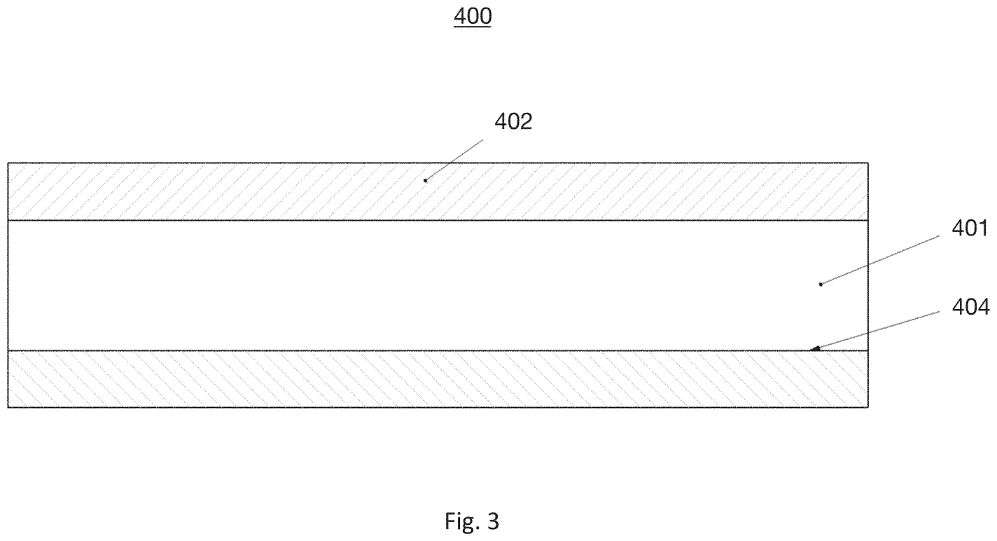

FIG. 3 is another example, medium 400 where the channel 401 is a rectangle stretching across the length of the medium 400.

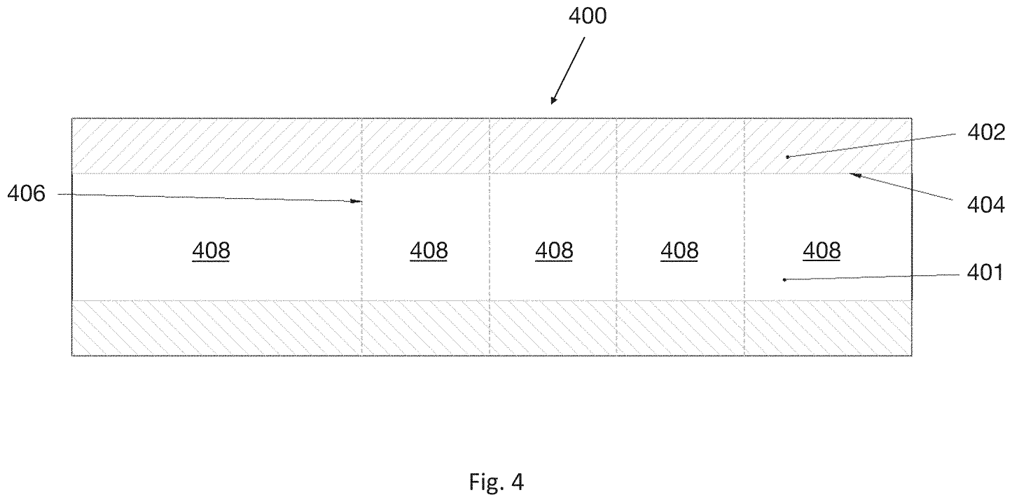

FIG. 4 is a medium 400 similar to the example of FIG. 3, but with break lines 406 defining multiple sections 408.

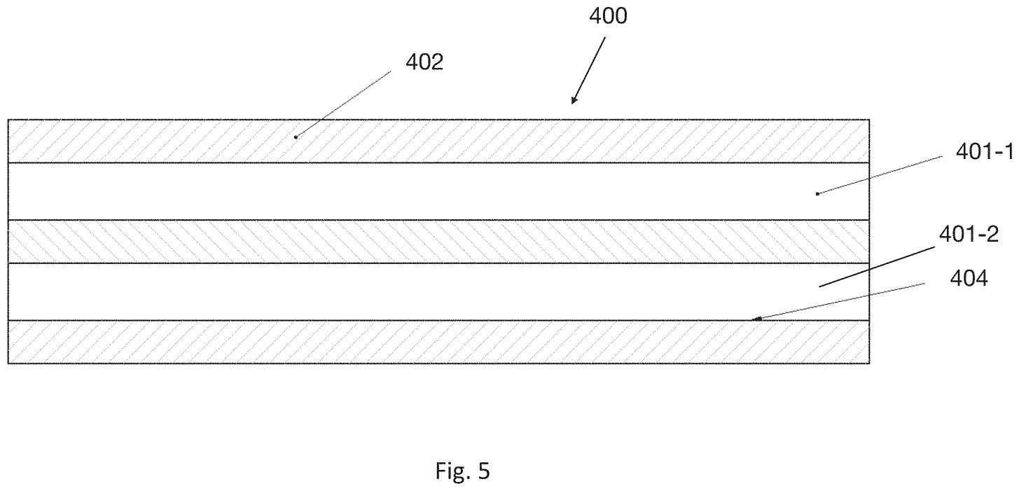

FIG. 5 is an example medium 400 with hydrophobic region 402 defining two parallel channels 401-1, 401-2.

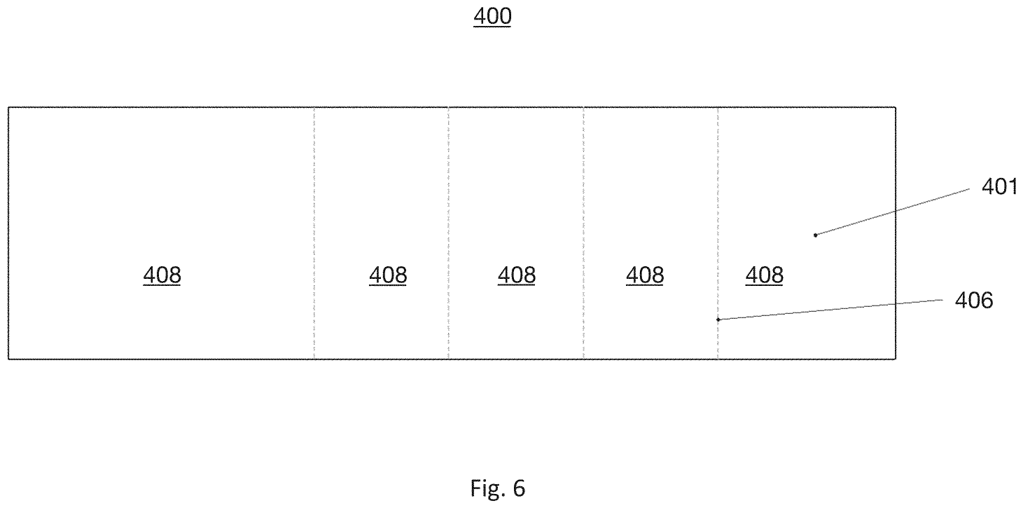

FIG. 6 is an implementation of the medium 400 with just break lines that define different sections 408, and without any hydrophobic region patterning.

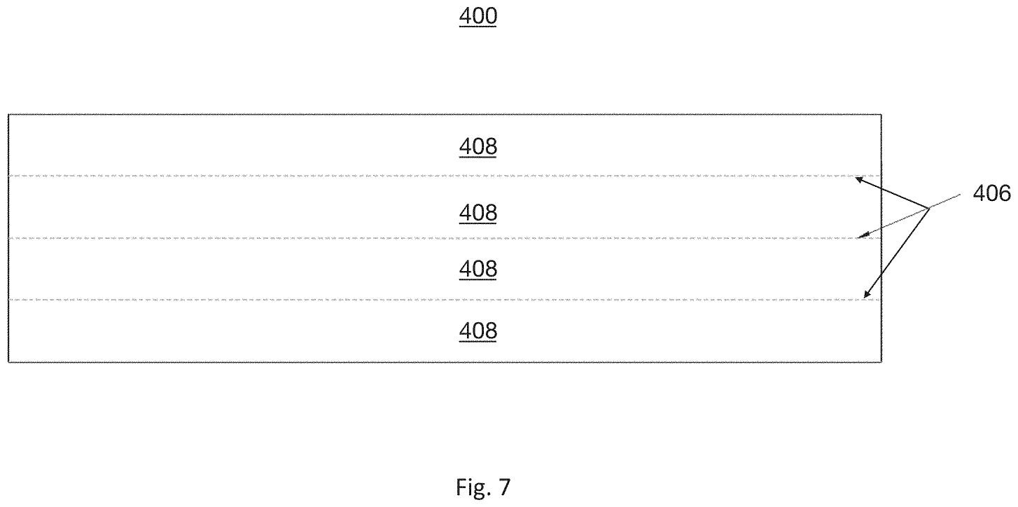

FIG. 7 shows an example similar to FIG. 6, but here the break lines 406 run lengthwise across the medium 400 to define four (4) sections 408.

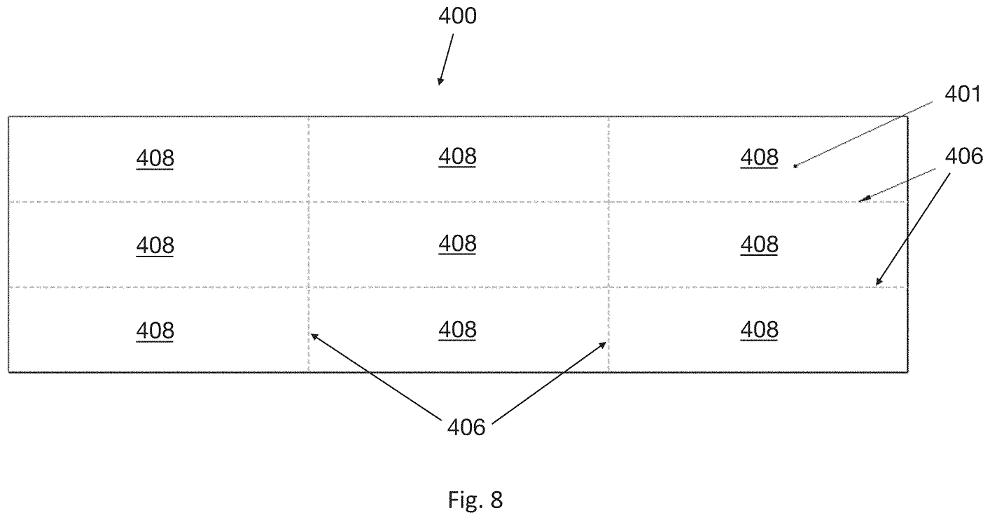

FIG. 8 is an example embodiment where the break lines 406 run both lengthwise and transverse across the channel 401. Here there are, for example, nine (9) different sections of the medium 400 are delineated.

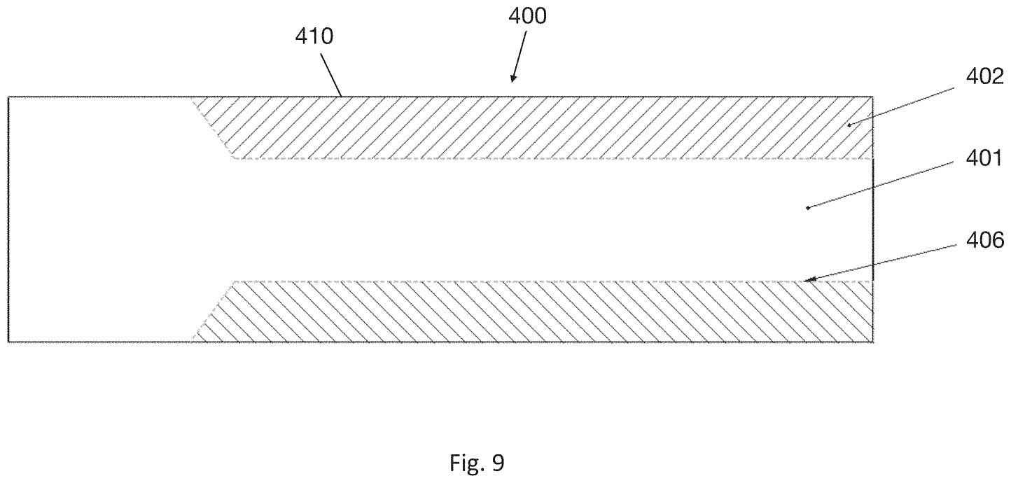

FIG. 9 is yet another example of medium 400 with break lines 406 formed along the edges of the channel 401, that is, at, near, or otherwise conformal to the edges of the hydrophobic region pattern 402.

In this and the other embodiments, the medium 400 may also be a lateral flow strip held in a housing 410 which is partially or fully formed from the hydrophobic region 402. break lines 406 allow the separation of the lateral flow strip from such a hydrophobic housing 410.

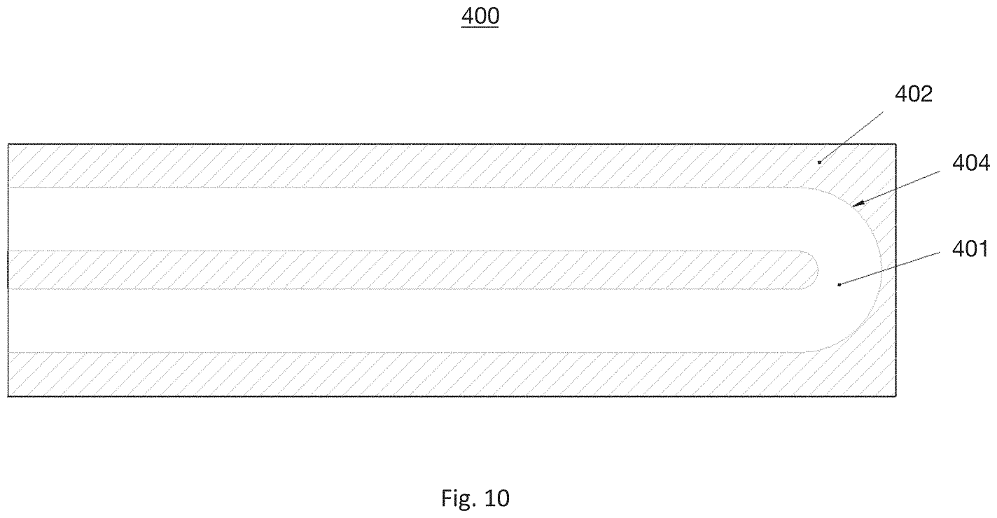

FIG. 10 is an example where the medium 400 includes a single channel 401 that follows a curved path.

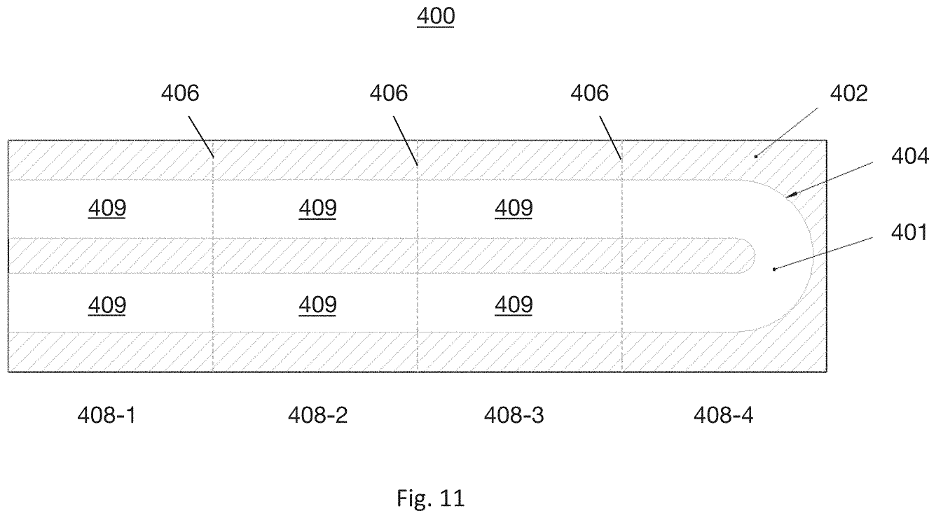

FIG. 11 is a similar implementation to FIG. 10 having a single channel 401 that follows a curved path, but with three (3) break lines 406 defining four (4) sections 408. Some of the sections 408-1, 408-2, 408-3 contain two (2) collection areas 409.

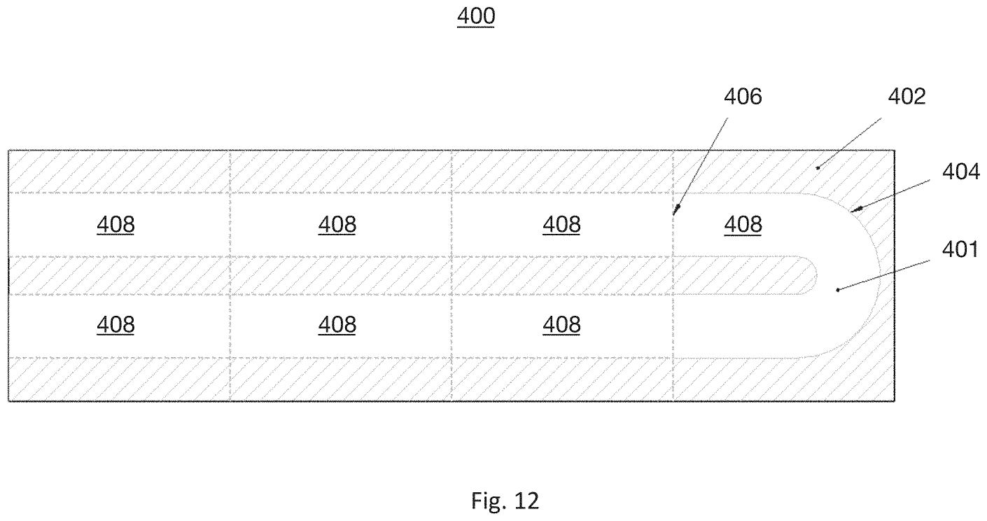

The embodiment of FIG. 12 is similar to FIG. 11, but has break lines 406 formed only on certain parts of the sides of the channel 401. Thus, when the medium 400 is broken along the break lines 406, it will provide sections 408 of a different size and shape than the FIG. 11 embodiment.

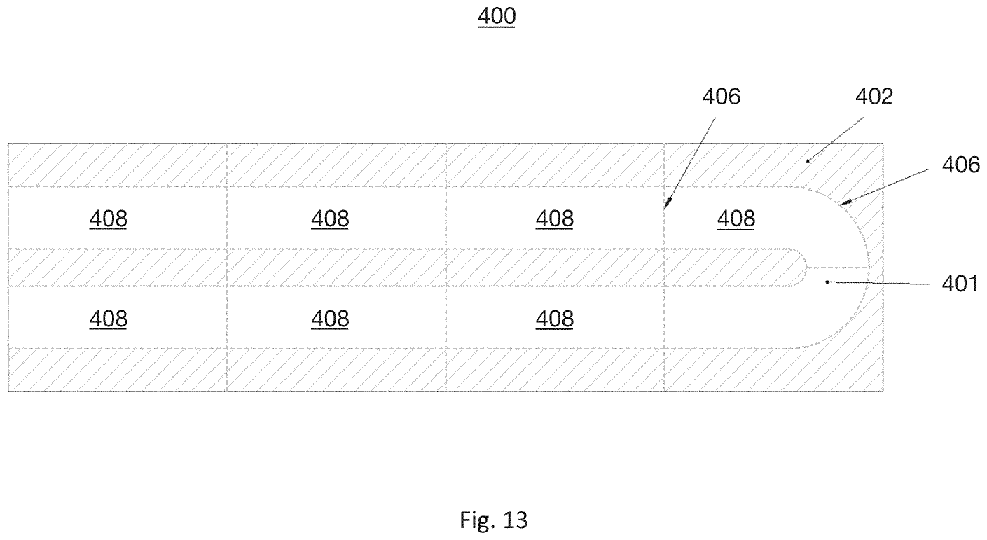

FIG. 13 is another example similar to FIG. 12 where the break lines follow the curved channel 401 along its entire length.

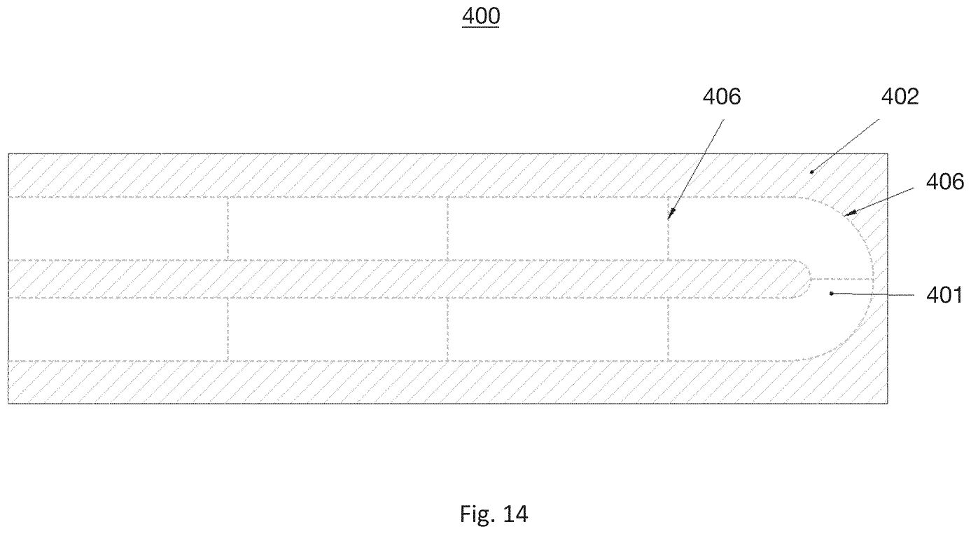

FIG. 14 is another arrangement where the medium 400 has been coated with a substance 402 such as a hydrophobic substance. The hydrophobic substance 402 directs the blood sample into eight sections formed in the channel 401. In this embodiment, the channel 401 may follow a curved path but other paths are possible. FIG. 14 also shows that the break lines 406 may not necessarily run to the edges of the channel.

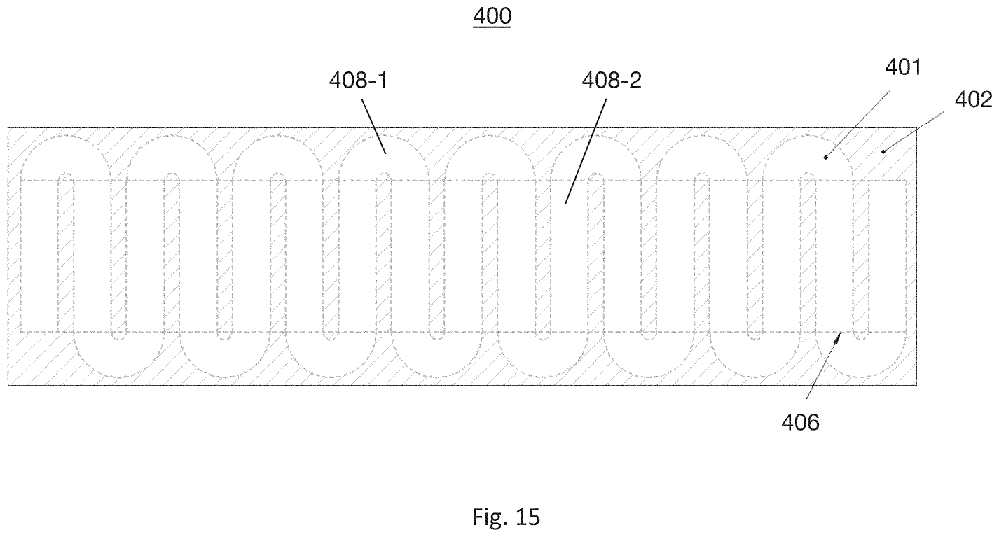

FIG. 15 is an example where a serpentine channel 401 runs the length of the medium 400, with break lines 406 defining several sections of a serpentine channel. Sections 408-1 and 408-2 may have different shapes and sizes. The different sections may be coated with various reagents, as with other embodiments.

The hydrophobic region regions may therefore define different types or shapes of fluid pathways for the channel(s) 401. Differently shaped and lengthened paths, such as the illustrated serpentine path, or other types of tortuous paths, may regulate and/or slow the speed of fluid movement through or along the medium 400. Slowing the speed of fluid sample movement may, in turn, allow the medium to more fully absorb the fluid, such as via a resulting slowed capillary action.

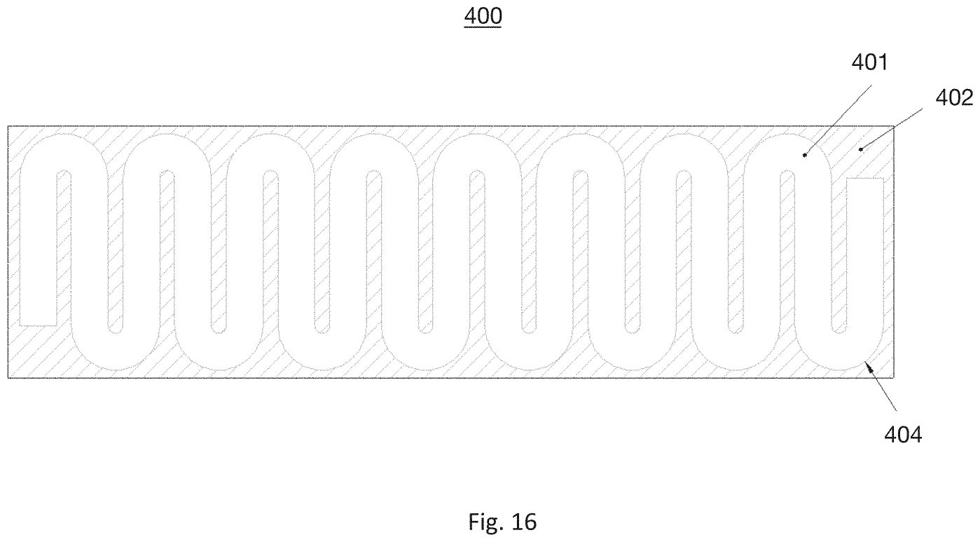

FIG. 16 is similar to FIG. 15, but without the break lines.

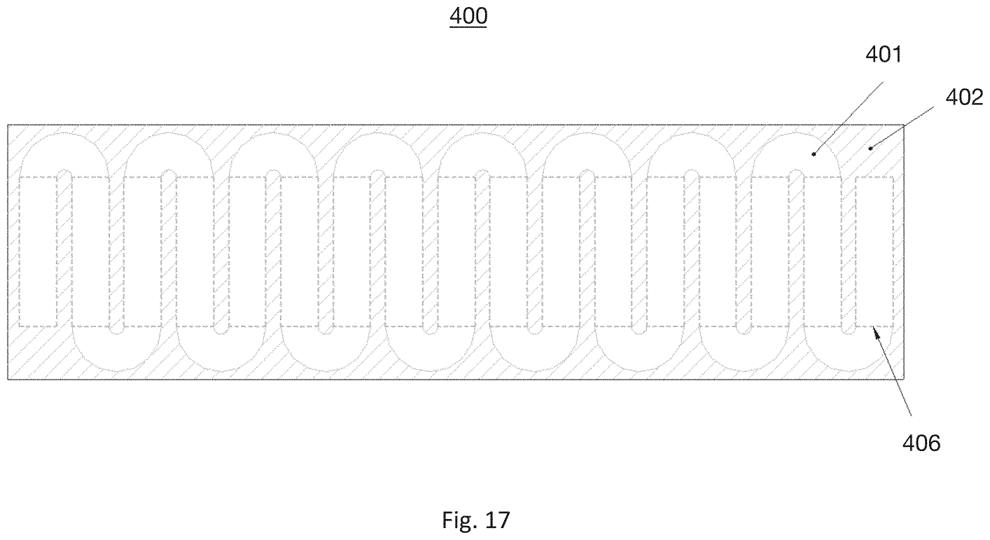

FIG. 17 is another arrangement of break lines with a serpentine channel 401.

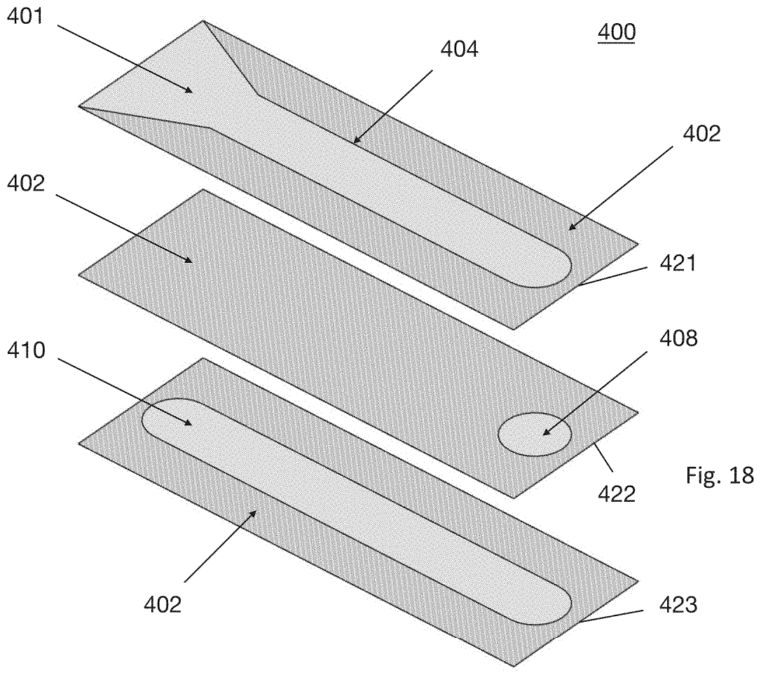

The embodiment of FIG. 18 is a “three-dimensional” implementation where the channel occupies more than one layer. In this example, the channel 401 defined by the hydrophobic region 402 starts on a top layer 421, and may be straight as shown, or serpentine, or follow other paths. The channel 401 on the top layer 421 defines a path to a location where the fluid may pass through a middle layer 422. The middle layer 422 here is mostly hydrophobic region 402, having only selected small area 408 or via through which the fluid can pass onto a bottom layer 423. The bottom layer 423, in turn, may also define a path 410 (which may be straight as shown, or serpentine, or follow other paths) bordered by hydrophobic region 402. Each of these layers 421, 422, 423 may be made of a different medium material or have different hydrophobic or hydrophilic treatments to direct fluid. Other three dimensional arrangements are possible, such as with different patterns of channels 401 and 410, additional vias 408, and more than three layers. The multiple layer embodiment may include break lines as described for the other embodiments.

FIG. 18 may also be used to define a sample collection well 430 that directs a sample to prefilter (such as disposed within the via 408) positioned over a bottom layer 423 that provides a lateral flow strip 410. This arrangement also allows for pretreatment of a sample with reagents contained in either channel 400 or 408 before the sample is directed to the lateral flow strip 410. hydrophobic region 402 keeps these layers and reagents physically separated from the sample so they may only be encountered in the intended order.

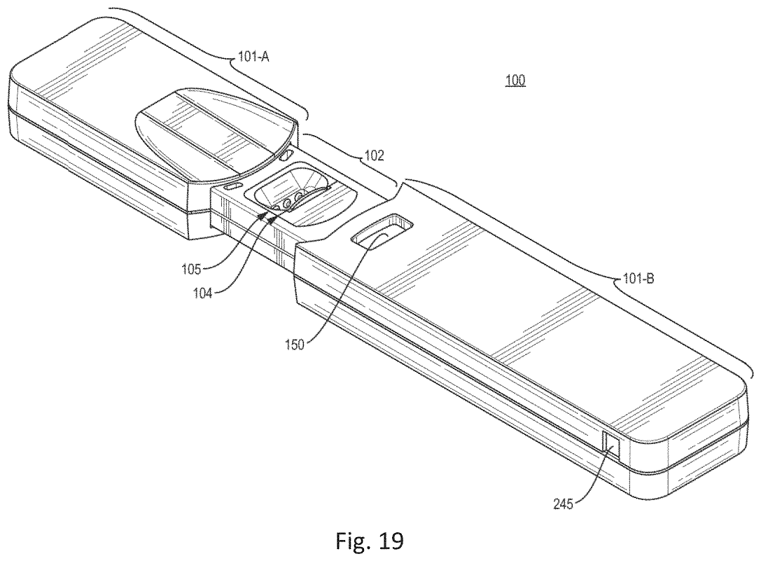

FIG. 19 is an example of a blood collection device 100 that may use any of the media 400 as described herein. However, there are other types of devices that can use the media 400 and take advantage of the same principles. Some example devices were described in a co-pending U.S. patent application Ser. 16/164,988 filed Oct. 19, 2018 for “Fluid Sample Collection Device”, the entire contents of which are hereby incorporated by reference.

The device 100 includes a two-piece housing 101 that supports and encloses a fluid sample port 102. The housing 101 includes a first housing piece 101-A and second housing piece 101-B. In this view, the housing is in the open position with the two housing pieces 101-A, 101-B spaced apart from one another, to provide access to the sample port 102. A sample collection well 104 and one or more capillaries 105 located adjacent the sample port 102 are partially visible in this figure. A window 150 in the housing permits a user to confirm the status of one or more portions of a fluid sample in the process of being collected and/or stored within the device 100.

The device 100 is initially presented in its open position, as per FIG. 18, to provide access to the well 104. A user, such as a patient herself or a health care professional, then uses a lancet to produce a blood sample such as from a finger tip. Drops of whole blood are then taken with the finger positioned near to, above, adjacent to, or even in contact with the well 104 or other parts of the sample port 102 to minimize blood spillage.

Blood is then eventually drawn into the rest of the device 100 in one or more different ways. As will be explained in more detail below for one embodiment, blood flows and/or is first drawn from the well 104 by one or more collection capillaries 105 adjacent to the sample port via capillary action. The capillaries may be visibly transparent so that the user can confirm that blood is being properly drawn into the device 100. The capillaries 105 can optionally be pre-coated with reagents such as heparin and/or EDTA for subsequent stabilization and preservation of the sample. The capillaries 105 can also have a known and predetermined volume, in which case the incoming sample is precisely metered. The collection capillaries 105 then direct the metered sample to a medium (such as any of the medium 400 described herein) inside the device housing 101.

The user, who can be the patient himself/herself or a healthcare professional, then manually closes the device 100 by pushing the two housing pieces 101-A, 101-B together, causing the sample to be deposited onto the medium 400.

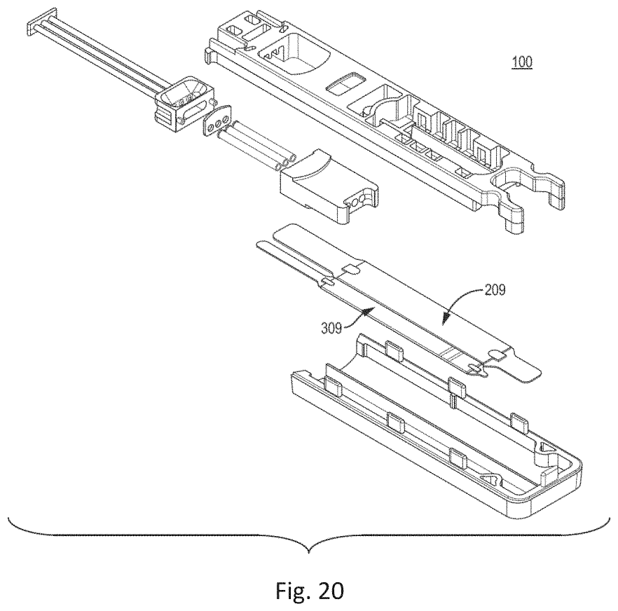

FIG. 20 is a more detailed, exploded view of the components of the device 100.

A backbone structure 203 provides a support for the housing pieces 101-A, 101-B, allowing them to slide back and forth, and thus to move the housing into the open or closed position.

The backbone 203 also supports other components of the device 100. For example, the backbone 203 provides a location for the sample collection port 102, a plunger rack 202, or a ribbed section 230 to support a desiccant tablet (not shown) to further dry the collected sample. The backbone 203 may also have tines at an end that provide a ratcheting closure 240, which is activated when the two housing pieces 101-A, 101-B are pushed together.

Capillaries 204 are inserted into and held in place by longitudinal holes in an inlay 252 piece. The capillaries and may be formed as a rigid tube of precisely defined volume, in which case they also serve a metering function. The capillaries 204 extract a defined quantity of blood by engagement with the blood in the sample collection port 102 through capillary action. The inlay 252 may fit into a hole 221 in backbone 203. The capillaries 204 can optionally be pre-coated with reagents, heparin, EDTA, or other substances.

One or more capillaries 204 may also store a predetermined amount of a liquid reagent. Such a reagent may then be dispensed together or in parallel with the blood sample when the housing is moved from the open to the closed position. However, reagents of other types may also be located in a storage region within the housing. The storage region (not designated in the Figures), may hold a first type of reagent such as a solid surface or substrate, and a second type being a liquid storage chamber, each of which are placed in the path of the blood sample collected by the device 100.

In one arrangement, the one or more plungers 202 firmly engage with the inner diameter of the capillaries 204, creating a shutoff that blocks off any excess blood sample while also pushing the metered sample volume to the subsequent downstream processing steps.

A base 206 may also fit into the backbone 203 to provide additional mechanical support for the medium 400 in the form of a blood collection membrane 209. The membrane-type medium may be supported and/or held in place by other components that assist with handling the membrane 209 when it is removed from the device 101 for processing by a laboratory.

This particular device 100 has two media—including both a collection membrane 209 and an immunoassay strip 309. The membrane 209 and strip 309 may be arranged in parallel. The collection membrane 209 receives and stores a blood sample exiting from some capillaries, and the immunoassay (or other test) strip 309 may receive and process a blood sample exiting from other capillaries.

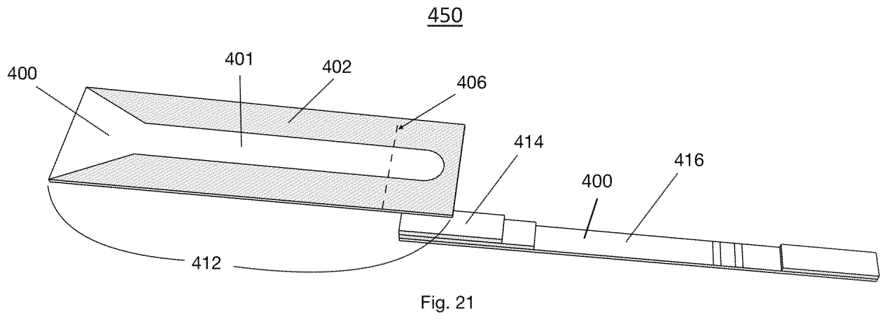

FIG. 21 is an exploded view of a device 450 that has a medium 400 formed of multiple layers of membrane. In this case, the medium 400 includes a first layer 412 that is a membrane with a hydrophobic section 402 and a second layer 416 that is a lateral flow strip located beneath the first layer 412. The hydrophobic section 402 creates a channel 401 to direct a fluid sample over a sample pad 414 of the lateral flow strip 416 located below. Channel 401 may be used to direct a sample into a housing (not shown) that holds these membranes in place. In addition, one or more break lines406 allow the membrane 412with the channel to tear away the portion not in contact with the lateral flow strip 416. This allows lateral flow strip 416 to be removed from the housing for analysis without removing all sections of the membrane 412 which may contain undesirable material such as red blood cells, or simply be anchored within the device. In some embodiments, the lateral flow strip 416 may itself contain further multiple strips or other collection medium 400. Still further additional layers can be added as ways of providing reagents, or directing the path of fluids, or for holding other components in place, and for other purposes.

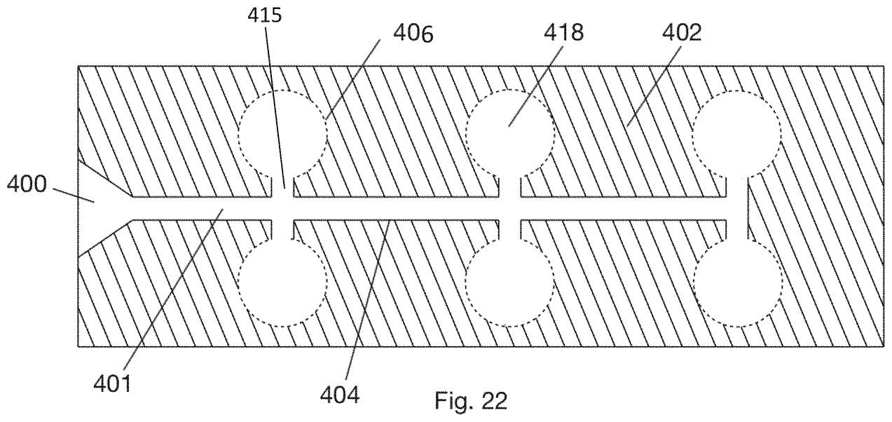

FIG. 22 shows a section of a membrane-type medium 400 that has a channel 401 that includes multiple branches 415 defined by a hydrophobic region 402. At the end of each branch 415 there is a circular-shaped area 418 bordered by break line 406 which allows the removal of the circular area 418 of membrane for analysis. These removable portions may come in a variety of shapes other than circular, and may be sized to ensure a desired volume of sample. Alternatively, these perforated areas 418 may serve as reaction wells that can be removed.

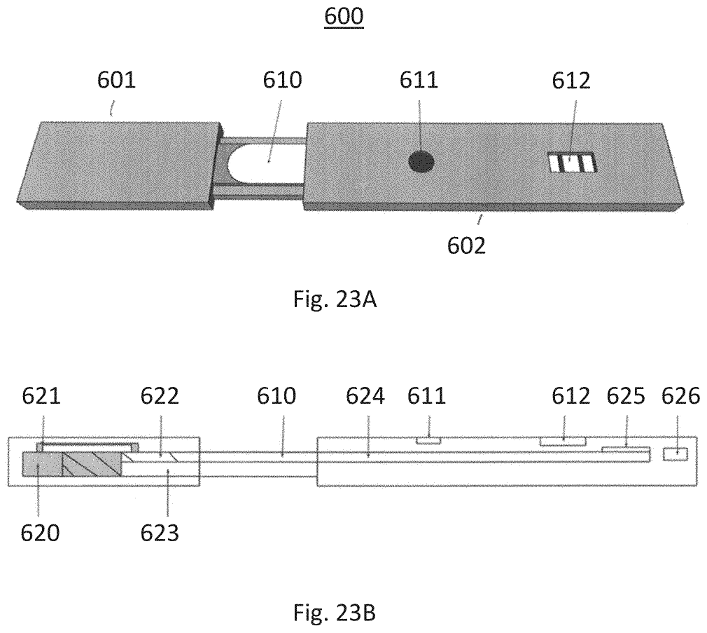

FIGS. 23A and 23B are another device 600 that uses hydrophobic principles to define a medium 400 that provides a lateral flow strip. This device 600 consists of a movable or removable cap 601 and a main body 602. A sample collection port 610 provides a location for collecting a blood sample, a fill window 411 provides visual feedback as to whether a sufficient amount of sample has been introduced into the device 600, and a results window 612 permits viewing a result area of the lateral flow strip.

In this embodiment:

620 is a liquid reagent reservoir;

621 is a fluid channel that connects the liquid reagent reservoir 621 with the sample collection port once the cap is placed on and/or slid inward to close the device;

622 is an empty region in the device that the sample collection port moves into when the device is closed;

623 is a rigid support underneath the lateral flow strip that extends into the liquid reagent portion of the housing;

624 is a lateral flow strip provided by a medium 400 contains one or more hydrophobic patterns and/or break lines as described in any of the embodiments above;

625 is a sample absorbent pad at the end of the lateral flow strip; and

626 is a desiccant tab.

Therefore, it should be understood that in light of the above, various modifications and additions may be made to the embodiments described herein without departing from the true scope of the inventions made.

1. A fluid sample collection device comprising:
a medium having a channel defined by at least one hydrophobic region; and
at least one break line intersecting the channel, and defining a predetermined area of the medium.

2. The device medium of claim 1 wherein at least one break line defines an area providing a predetermined volume for collection of a fluid sample.

3. The device medium of claim 1 further comprising two or more break lines that define corresponding multiple areas of the medium coated with reagents, and with at least one selected reagent coating a selected area being different from another reagent coating another area.

4. The device of claim 1 wherein the hydrophobic region further defines one or more fluid pathways.

5. The device of claim 1 wherein the hydrophobic region regulates a speed of fluid movement.

6. The device of claim 5 wherein the hydrophobic region defines a tortuous path to slow fluid movement through the device.

7. The device of claim 5 wherein the hydrophobic region slows t fluid movement, in turn partially saturating the medium to slow capillary action.

8. The device of claim 1 wherein the medium further comprises multiple layers.

9. The device of claim 8 wherein a first layer is a first membrane that collects and directs a sample to a second layer by way of a channel defined by the hydrophobic region.

10. The device of claim 8 wherein one of the layers is a lateral flow strip.

11. The device of claim 8 wherein one of the layers contains a reagent.

12. The device of claim 8 wherein one of the layers contains a filtering medium.

13. The device of claim 8 wherein one of the layers redirects a fluid flow via one or more of hydrophobic or hydrophilic materials.

14. The device of claim 1 wherein the medium comprises a membrane.

15. The device of claim 1 wherein the medium comprises a microstructured environment.

16. A fluid collection device comprising:
a housing with a first position and a second position;
a sample port to collect a biological sample which is open in the first position;
a structured microenvironment designed to absorb and meter the biological sample; and
a fluid control system disposed within the housing that transfers biological sample from the sample port to the structured microenvironment when moved from the first position to the second position.

17. A device of claim 16 where the structured microenvironment is a membrane.

18. A device of claim 16 where the structured microenvironment separates red cells from plasma.

19. A device of claim 16 where fluid in the sample port is treated with a reagent before reaching the structured microenvironment.

20. A device of claim 16 where the structured microenvironment is configured to collect two or more portions with similar volumes.

21. A device of claim 20 where the portions are connected to a single flow path and easily separated.
